(19) United States
(12) Patent Application Publication
(10) Pub. No.: US 2020/0171082 A1
(43) Pub. Date: Jun. 4, 2020

(54) QUANTIFYING SLFN11 PROTEIN FOR OPTIMAL CANCER THERAPY

(71) Applicants: NantOmics, LLC, Culver City, CA (US); MEMORIAL SLOAN-KETTERING CANCER CENTER, New York, NY (US)

(72) Inventors: Fabiola CECCHI, Potomac, MD (US); Sarit SCHWARTZ, Rockville, MD (US); Todd HEMBROUGH, Gaithersburg, MD (US); Charles Michael RUDIN, New York, NY (US); John Thomas POIRIER, New York, NY (US)

(21) Appl. No.: 16/623,972

(22) PCT Filed: Jun. 20, 2018
(86) PCT No.: PCT/US2018/038546

§ 371 Date: Dec. 18, 2019

Related U.S. Application Data
(60) Provisional application No. 62/522,670, filed on Jun. 20, 2017.

(51) Int. Cl. A61K 33/243; C12Q 1/37; C12Q 1/6886; G01N 33/68

(52) U.S. Cl. CPC A61K 33/243; C12Q 1/37; A61K 45/06; G01N 33/6848; C12Q 1/6886

(57) ABSTRACT

Methods are provided for identifying whether a tumor, and especially a lung tumor, will be responsive to treatment with a therapeutic regimen that contains a platinum-based agent such as cisplatin and optionally contains a taxane. A specific Schlafen family member 11 (SLFN11) fragment peptide is precisely detected and quantitated by spectrometry directly in lung cancer cells collected from lung tumor tissue obtained from a cancer patient. Comparison to reference levels determines if the cancer patient will respond positively or negatively to treatment with the chemotherapeutic agents taxane plus a platinum-based agent such as cisplatin.

This application claims priority to U.S. Provisional Application No. 62/522,670, titled “Quantifying SLFN11 Protein For Optimal Cancer Therapy,” filed Jun. 20, 2017, the contents of which are hereby incorporated by reference in their entirety.

Improved methods are provided for treating cancer patients. Tumor tissue from a patient is assayed and the results of the assay are used to select an improved or optimal treatment regimen that is administered to the patient.

### BACKGROUND

Currently, no predictive biomarkers are approved to indicate outcomes for treatment of cancer patients with standard chemotherapy. The Schlafen family member 11 (SLFN11) protein is widely reported as necessary for sensitivity to DNA-damaging chemotherapy agents. Epigenetically-mediated suppression of SLFN11 is associated with poor response to platinum-based agents in patients with ovarian and lung cancer. In addition, pre-clinical lung cancer models suggest that SLFN11 expression may be a useful biomarker of response to cisplatin, PARP inhibitors, and topoisomerase inhibitors.

Tumor expression of SLFN11 is currently assessed by immunohistochemistry, RNA expression or DNA methylation. No standardized methods exist, however, to effectively measure the amount of SLFN11 protein in patient tumor cells. Methods are provided for a mass spectrometry-based assay that precisely quantifies the SLFN11 protein in archived samples of early-stage lung cancer patients treated with taxane plus platinum (TP) and correlates quantitative levels of the SLFN11 protein with overall patient survival.

Lung cancer is the most common cause of cancer-related death in men and second most common in women after breast cancer. Overall, 17.4% of people in the United States diagnosed with lung cancer survive five years after the diagnosis, while outcomes on average are worse in the developing world. Platinum-based DNA damaging chemotherapy agents can prolong survival of lung cancer patients. The chemotherapy drug cisplatin is a standard treatment for patients with many different types of cancer, including lung cancer. Taxol-based microtubule disrupting chemotherapy agents can also prolong lung cancer patient survival. Paclitaxel is a microtubule disrupting agent and is generally considered to be one of the most commonly-used chemotherapy agent for many different types of cancers, including lung cancer.

Platinum-based chemotherapy agents cause crosslinking of DNA as monoadduct, interstrand crosslinks, intrastrand crosslinks or DNA protein crosslinks. These agents act mostly are provided n the adjacent N-7 position of guanine, forming a 1, 2-intrastrand crosslink. The resultant crosslinking inhibits DNA repair and/or DNA synthesis in cancer cells.

The SLFN11 protein has multiple functions. One is to bind to and sequester tRNAs which prevents their maturation via post-transcriptional processing and accelerates their deacylation, thus preventing protein synthesis. SLFN11 does not inhibit reverse transcription, integration or production and nuclear export of viral RNA. In addition, SLFN11 may play a role in cell cycle arrest and/or induction of apoptosis in response to exogenously induced DNA damage. Thus, higher levels of SLFN11 protein in tumor cells can help to prevent protein synthesis as well as promote apoptosis of tumor cells after DNA damage induced by a chemotherapy agent, such as a platinum-based chemotherapy agent.

### SUMMARY OF THE INVENTION

Methods are provided for treating a patient suffering from lung cancer, including the steps of (a) quantifying by mass spectrometry the level of a specified SLFN11 fragment peptide in a protein digest, such as a protease digest, prepared from a tumor sample obtained from the patient and calculating the level of the SLFN11 fragment peptide in the sample; (b) comparing the level of the SLFN11 fragment peptide to a reference level, and (c) treating the patient with a therapeutic regimen, where the regimen includes an effective amount of a combination of a taxane chemotherapeutic agent plus a platinum-based agent when the level of the SLFN11 fragment peptide is higher than the reference level, or (d) treating the patient with a therapeutic regimen that does not include an effective amount of a combination of a taxane chemotherapeutic agent plus a platinum-based agent when the level of the SLFN11 fragment peptide is below the reference level. The reference level may be 100 amol/μg., +/−95 amol/μg, +/−75 amol/μg, +/−50 amol/μg, or +/−25 amol/μg, of biological sample protein analyzed. The protein digest may be a trypsin digest. The tumor sample may be a cell, collection of cells, or a solid tissue and may be formalin-fixed and, optionally, paraffin embedded.

The mass spectrometry may be tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry, and the mode of mass spectrometry used may be Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

Advantageously the specified SLFN11 peptide has the amino acid sequence YTPESLWR (SEQ ID NO:1) and is quantified by comparing to a spiked internal standard peptide of known amount, where both the native peptide in the biological sample and the internal standard peptide have the amino acid sequence YTPESLWR (SEQ ID NO:1). Advantageously, the internal standard peptide is isotopically labeled with, for example, one or more heavy stable isotopes such as ¹⁸O, ¹⁷O, ¹⁵N, ¹³C, ²H and combinations of these isotopes.

These methods can be used to inform the treatment decision about which chemotherapy agent, or agents, is used for treating a cancer patient. In addition, these methods can be combined with detecting and quantitating other peptides from other proteins in a multiplex format so that the treatment decision about which agent, or agents, used for treatment is based upon specific levels of the specified SLFN11 fragment peptide or SLFN11 protein in combination with other peptides/proteins in the biological sample.

### BRIEF DESCRIPTION OF THE DRAWING

FIG. 1 shows a Progression Free Survival (PFS) curve for patients where patients whose tumor tissue express levels of the SLFN11 protein >100 amol/μg of tumor cell protein have statistically significant longer PFS than patients whose tumor cells express >100 amol/μg of tumor cell protein when treated with taxane plus a platinum-based agent. Results are shown as probability (0-100%) for overall survival in days after initiation of treatment. The Mantel-Cox log-rank (p=0.0295) and Gehan-Breslow-Wilcoxon (p=0.0281) tests were used for survival comparisons and both indicate statistical significance.

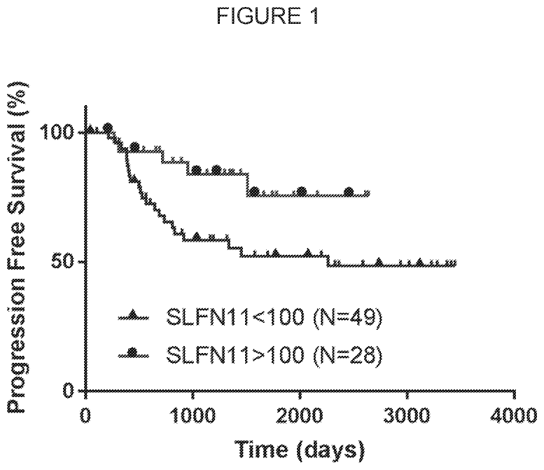

### DETAILED DESCRIPTION

Improved methods of treating cancer, and particularly lung cancer, are provided. The presence and/or quantitative levels of SLFN11 protein expression in cells within tumor tissue is determined and the measured levels are used to guide a treatment regimen that provides an improved outcome for the treated patient. More specifically, a specified peptide derived from a subsequence of the full-length SLFN11 protein is measured in a protein digest of tumor tissue from the patient, such as formalin-fixed tissue. If expression of the SLFN11 protein is above a specified quantitative level, the patient is treated with a therapeutic regimen that includes a platinum-based chemotherapeutic agent such as cisplatin, and/or other drugs that function similarly to platinum-based drugs. The therapeutic regimen may optionally contain a taxane drug. Alternatively, if the SLFN11 protein level is below the specified quantitative level, the patient is treated with an alternative therapeutic regimen that does not include a platinum-based chemotherapeutic agent such as cisplatin, or other drugs that function similarly as a platinum-based chemotherapeutic agent such as cisplatin.

The SLFN11 peptide advantageously is detected using mass spectrometry-based Selected Reaction Monitoring (SRM), also referred to as Multiple Reaction Monitoring (MRM), referred to herein as an SRM/MRM assay. An SRM/MRM assay is used to detect the presence of, and quantitatively measure, the amount of the specified SLFN11 fragment peptide directly in cells procured from cancer patient tissue, such as, for example formalin fixed cancer tissue. Each molecule of SLFN11 fragment peptide is derived from one molecule of SLFN11 protein and therefore measurement of the amount of the specific peptide allows quantitation of the amount of intact SLFN11 protein in the tumor sample. Specific and optimized therapeutic agents and treatment strategies can be used to treat an individual cancer patient's disease based on how much of the SLFN11 protein is detected in their cancer cells.

More specifically, methods are provided for determining if a cancer patient will clinically respond in a favorable manner to the therapeutic cancer agent taxane plus platinum (TP). The quantitative level of the SLFN11 protein in a tumor sample or samples from the patient are measured. The sample is advantageously formalin-fixed and may be paraffin embedded. The SRM/MRM assay advantageously measures a specified SLFN11 peptide fragment, and particular characteristics about the peptide, where the preferred peptide has the sequence YTPESLWR (SEQ ID NO:1). Surprisingly it has been found that this peptide can be reliably detected and quantitated in digests prepared from formalin-fixed, paraffin embedded (“FFPE”) samples of tumor tissue. See U.S. patent application Ser. No. 13,993,045, the contents of which are hereby incorporated by reference in their entirety.

The SRM/MRM assay can be used to measure these peptides directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue reagents and protocol available from Expression Pathology Inc. (Rockville, Md.).

The most widely and advantageously available form of tissue, and cancer tissue, from cancer patients is formalin fixed, paraffin embedded tissue. Formaldehyde/formalin fixation of surgically removed tissue is by far and away the most common method of preserving cancer tissue samples worldwide and is the accepted convention in standard pathology practice. Aqueous solutions of formaldehyde are referred to as formalin. “100%” formalin consists of a saturated solution of formaldehyde (about 40% by volume or 37% by mass) in water, with a small amount of stabilizer, usually methanol, to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature. Thus molecular analytical methods to analyze formalin fixed cancer tissue will be the most accepted and heavily utilized methods for analysis of cancer patient tissue.

Results from the SRM/MRM assay can be used to correlate accurate and precise quantitative levels of the SLFN11 protein within the specific cancer of the patient from whom the tissue was collected and preserved, including lung, colon, skin, and brain cancer tissue. Advantageously, the levels of SLFN11 protein are measured in a tumor sample from lung cancer. This not only provides diagnostic information about the cancer, but also permits a physician or other medical professional to determine appropriate therapy for the patient. In this case, utilizing this assay can provide information about specific levels of SLFN11 protein expression in cancer tissue and whether or not the patient from whom the cancer tissue was obtained will respond in a favorable way to therapy with anti-cancer therapeutic agents that include platinum-based agents such as cisplatin, and/or potentially other similar drugs designed to specifically damage DNA in tumor cells.

Treating cancer patients with platinum-based agents such as cisplatin is one of the most common and effective strategies for preventing cancer from growing and thus prolonging the lives of cancer patients, especially lung cancer patients. The SLFN11 protein is a protein that promotes cell apoptosis after DNA damage; more specifically it induces apoptosis after the type of DNA damage inflicted by the platinum-based therapeutic agents such as cisplatin, and other similar DNA damaging agents. Accordingly, if there is SLFN11 protein present in a tumor cell that is being treated with cisplatin, and/or other similar agents, the presence of the DNA damage promotes the SLFN11 protein to induce apoptosis of the tumor cell, thus killing the tumor cell. It therefore is useful for a clinician to know if the SLFN11 protein is present in a patient's cancer cells because the effects of platinum-based chemotherapeutic agents, such as cisplatin, will be enhanced and the cancer patient will likely respond more favorably to platinum-based chemotherapeutic agents such as cisplatin, and/or other similar DNA damaging agents.

Presently the most widely-used and applied methodology to determine protein presence in cancer patient tissue, especially FFPE tissue, is immunohistochemistry (IHC). IHC methodology utilizes an antibody to detect the protein of interest. The results of an IHC test are most often interpreted by a pathologist or histotechnologist. This interpretation is subjective and does not provide quantitative data that can be predictive of taxane plus platinum (TP) sensitivity or resistance.

Research from other IHC assays, such as the Her2 IHC test, suggest the results obtained from such tests may be wrong. This is probably because different labs have different rules for classifying positive and negative IHC status. Each pathologist running the tests also may use different criteria to decide whether the results are positive or negative. In most cases, this happens when the test results are borderline, meaning that the results are neither strongly positive nor strongly negative. In other cases, tissue from one area of cancer tissue can test positive while tissue from a different area of the cancer tests negative. Inaccurate IHC test results may mean that patients diagnosed with cancer do not receive the best possible care. If all or part of a cancer is positive for a specific target oncoprotein but test results classify it as negative, physicians are unlikely to recommend the correct therapeutic treatment, even though the patient could potentially benefit from those agents. If a cancer is oncoprotein target negative but test results classify it as positive, physicians may recommend a specific therapeutic treatment, even though the patient is unlikely to get any benefits and is exposed to the agent's secondary risks.

In light of this there is great clinical value in the ability to correctly evaluate accurate quantitative levels of the SLFN11 protein in tumors, especially lung tumors, so that the patient will have the greatest chance of receiving the correct chemotherapy that may or may not include treatment with platinum-based agents such as cisplatin, and/or other similar DNA damaging agents.

Determining quantitative levels of a specified SLFN11 fragment peptide can be performed in a mass spectrometer using the SRM/MRM methodology, whereby the SRM/MRM signature chromatographic peak area of a peptide is determined within a complex peptide mixture present in a Liquid Tissue lysate (see U.S. Pat. No. 7,473,532, as described above). Quantitative levels of the SLFN11 protein are then determined by the SRM/MRM methodology whereby the SRM/MRM signature chromatographic peak area of an individual specified peptide from the SLFN11 protein in one biological sample is compared to the SRM/MRM signature chromatographic peak area of a known amount of a “spiked” internal standard for the individual SLFN11 fragment peptide.

In one embodiment, the internal standard is a synthetic version of the same exact SLFN11 fragment peptide where the synthetic peptide contains one or more amino acid residues labeled with one or more heavy isotopes. Such isotope labeled internal standards are synthesized so that mass spectrometry analysis generates a predictable and consistent SRM/MRM signature chromatographic peak that is different and distinct from the native SLNF11 fragment peptide chromatographic signature peaks and which can be used as a comparator peak. When the internal standard is spiked in known amounts into a protein or peptide preparation from a biological sample and analyzed by mass spectrometry, the SRM/MRM signature chromatographic peak area of the native peptide is compared to the SRM/MRM signature chromatographic peak area of the internal standard peptide, and this numerical comparison indicates either the absolute molarity and/or absolute weight of the native peptide present in the original protein preparation from the biological sample. Quantitative data for fragment peptides are displayed according to the amount of protein analyzed per sample.

Additional information beyond the peptide sequence may be used to develop the SRM/MRM assay for a specified SLFN11 fragment peptide. That additional information is used to direct the mass spectrometer, (e.g., a triple quadrupole mass spectrometer) to perform the correct and focused analysis of the specified SLFN11 fragment peptide. An SRM/MRM assay advantageously is performed on a triple quadrupole mass spectrometer, which is the most suitable instrument for analyzing a single isolated target peptide within a very complex protein lysate that may consist of hundreds of thousands to millions of individual peptides from all the proteins contained within a cell. Although the most advantageous instrument platform for SRM/MRM assay is typically considered to be a triple quadrupole instrument, SRM/MRM assays can be developed and performed on any type of mass spectrometer, including a MALDI, ion trap, ion trap/quadrupole hybrid, or triple quadrupole. The additional information about target peptides in general, and in particular about a specified SLFN11 peptide, may include one or more of the mono isotopic mass of each peptide, its precursor charge state, the precursor m/z value, the m/z transition ions, and the ion type of each transition ion. The sequence of the specified peptide of SLFN11 used in the methods described herein is YTPESLWR (SEQ ID NO: 1).

To determine an appropriate reference level for SLFN11 quantitation, tumor samples are obtained from a cohort of patients suffering from cancer, in this case lung cancer. The lung tumor samples are formalin-fixed using standard methods and the level of SLFN11 in the samples is measured using the methods as described above. The tissue samples may also be examined using IHC and FISH using methods that are well known in the art. The patients in the cohort are treated with a combination of the therapeutic agents taxane plus cisplatin. The response of the patients is measured using methods that are well known in the art, for example by recording the progression-free survival and overall survival of the patients at time intervals after treatment. A suitable reference level can be determined using statistical methods that are well known in the art, for example by determining the lowest p value of a log rank test.

Once a reference level has been determined it can be used to identify those patients whose SLFN11 expression levels indicate that they may likely benefit from a combination therapeutic regimen that includes a platinum-based agent such as cisplatin. The skilled artisan will recognize that cisplatin is also used as part of a treatment regimen that utilizes additional drugs or combinations of drugs, such as in combination with taxane. Therapeutic regimens for treating lung cancer are known in the art.

For those patients where protein expression as measured using the methods described herein indicates that treatment with a combination therapeutic regimen that includes a platinum-based agent such as cisplatin is unlikely to be effective, an alternative therapeutic regimen may be used. Other therapeutics regimens include surgery (including wedge resection, segmental resection, lobectomy and pneumonectomy), radiation therapy, and targeted drug therapy (such as treatment with Afatinib (Gilotrif), Bevacizumab (Avastin), Ceritinib (Zykadia), Crizotinib (Xalkori), Erlotinib (Tarceva), Nivolumab (Opdivo) and Ramucirumab (Cyramza)).

Levels of the SLFN11 protein in patient tumor samples are typically expressed in amol/μg, although other units can be used. The skilled artisan will recognize that a reference level can be expressed as a range around a central value, for example, +/−250, 150, 100, 95, 50 or 25 amol/μg. In the specific example described in detail below a suitable reference level for the SLFN11 protein was found to be 100 amol/μg which is the lower limit of quantitation. However, the skilled artisan will recognize that levels higher or lower than these reference levels can be selected based on clinical results and experience.

Because both nucleic acids and protein can be analyzed from the same Liquid Tissue biomolecular preparation it is possible to generate additional information about disease diagnosis and drug treatment decisions from the nucleic acids in same sample upon which proteins were analyzed. For example, if the SLFN11 protein is expressed by certain cells at increased/decreased levels when assayed by SRM, the data can provide information about the state of the cells and their potential for uncontrolled growth, choice of optimal therapy, and potential drug resistance. At the same time, information about the status of genes and/or the nucleic acids and proteins they encode (e.g., mRNA molecules and their expression levels or splice variations) can be obtained from nucleic acids present in the same Liquid Tissue biomolecular preparation.

Nucleic acids can be assessed simultaneously to the SRM analysis of proteins, including the SLFN11 protein. In one embodiment, information about the SLFN11 protein and/or one, two, three, four or more additional proteins may be assessed by examining the nucleic acids encoding those proteins. Those nucleic acids can be examined, for example, by one or more, two or more, or three or more of sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

### Example: Determination of a Predictive Value of SLFN11 Protein Expression for Combination Therapy Comprising Taxane Plus Platinum-Based Drugs in a Population of Lung Cancer Patients

Tumor expression of SLFN11 was measured by mass spectrometry to quantify SLFN11 protein in archived samples of early-stage lung cancer patients treated with taxane plus platinum (TP). SLFN11 expression levels were correlated with patient survival.

Methods: Archived formalin-fixed, paraffin-embedded tissue sections were obtained from patients with lung cancers of multiple subtypes that were treated with taxane plus platinum-based agents (n=594). A board-certified pathologist marked the tumor areas, which were microdissected and the tissue was solubilized using the Liquid Tissue protocol and reagents (Expression Pathology, Inc., Rockville, Md.). In each liquefied tumor sample, 60 protein biomarkers including SLFN11 were quantified with selected reaction monitoring mass spectrometry. Patients were stratified by a SLFN11 cutoff of 100 amol/μg, based on the proteomic assay's limit of quantification. Survival outcomes were assessed with Kaplan-Meier and Mantel-Cox log-rank analyses.

Results: Among 86 TP-treated lung cancer patients, those with SLFN11 protein levels above the cutoff (n=51) had better progression-free survival (PFS) than patients with SLFN11 levels below the cutoff (HR: 2.26; 95% CI: 1.08-4.72; p=0.052). Similar differences in PFS were found in the subset of patients with NSCLC (n=77) (HR: 2.79; 95% CI: 1.29-6.05; p=0.030). Differences in overall survival by SLFN11 expression were not statistically significant. In a group of untreated patients (n=134), there were no differences in PFS between patients with high and low expression of SLFN11.

Conclusions: Mass spectrometric evaluation of SLFN11 retrospectively identified responders to platinum-containing chemotherapy and could be used to predict response. Multiplexed proteomics can quantitate SLFN11 simultaneously with other therapeutically relevant proteins (eg, HER2, ALK, ROS1) to inform therapy selection at initial diagnosis and upon relapse.

1. A method of treating a patient suffering from lung cancer comprising:
(a) quantifying a level of a SLFN11 fragment peptide in a protein digest prepared from a tumor sample obtained from the patient and calculating a level of the SLFN11 fragment peptide in said sample by mass spectrometry;
(b) comparing the level of said SLFN11 fragment peptide to a reference level, and
(c) treating the patient with a therapeutic regimen comprising an effective amount of a platinum-based agent when the level of the SLFN11 fragment peptide is higher than said reference level, and
(d) treating the patient with a therapeutic regimen that does not comprise an effective amount of a platinum-based agent when the level of the SLFN11 fragment peptide is below said reference level.

2. The method of claim 1 wherein the therapeutic regimen in step (c) further comprises a taxane.

3. The method of claim 1 wherein said reference level of the SLFN11 fragment peptide is 100 amol/μg, +/−95 amol/μg, of tumor sample protein analyzed.

4. The method of claim 1 wherein said reference level is 100 amol/μg, +/−75 amol/μg, of tumor sample protein analyzed.

5. The method of claim 1 wherein said reference level is 100 amol/μg, +/−50 amol/μg, of tumor sample protein analyzed.

6. The method of claim 1 wherein said reference level is 100 amol/μg, +/−25 amol/μg, of tumor sample protein analyzed.

7. (canceled)

8. The method of claim 1, wherein said protein digest comprises a protease digest.

9. The method of claim 7, wherein said protein digest comprises a trypsin digest.

10. The method of claim 1, wherein the mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

11. The method of claim 10, wherein a mode of mass spectrometry used is Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

12. The method of claim 1, wherein the SLFN11 fragment peptide has the amino acid sequence as set forth as SEQ ID NO:1.

13. The method of claim 1 wherein the tumor sample is a cell, collection of cells, or a solid tissue.

14. The method of claim 13, wherein the tumor sample is formalin fixed solid tissue.

15. The method of claim 14, wherein the formalin fixed solid tissue is paraffin embedded tissue.

16. The method of claim 1, wherein quantifying the SLFN11 fragment peptide comprises determining an amount of the SLFN11 fragment peptide in said tumor sample by comparing to a spiked internal standard peptide of known amount, wherein both the SLFN11 fragment peptide in said tumor sample and the spiked internal standard peptide corresponds to SEQ ID NO:1.

17. The method of claim 16, wherein the spiked internal standard peptide is an isotopically labeled peptide.

18. The method of claim 17, wherein the isotopically labeled spiked internal standard peptide comprises one or more heavy stable isotopes selected from ¹⁸O, ¹⁷O, ¹⁵N, ¹³C, ²H and a combination thereof.

19. The method of claim 16, wherein quantifying said SLFN11 fragment peptide is combined with quantitating other peptides from other proteins in multiplex.


SEQUENCE LISTING (1 sequence)

SEQ ID NO: 1 - protein, 8 aa, Homo sapiens
YTPESLWR